(19) United States
(12) Patent Application Publication
(10) Pub. No.: US 2005/0074404 A1
(43) Pub. Date: Apr. 7, 2005

(54) Methods for avoiding renal injury

(75) Inventor: Michael D. Aleo, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/452,489

(22) Filed: May 30, 2003

Related U.S. Application Data
(60) Provisional application No. 60/384,728, filed on May 31, 2002.

(51) Int. Cl. G01N 33/53; A61K 49/00

(52) U.S. Cl. 424/9.1; 435/7.1

(57) ABSTRACT

The invention relates to methods for avoiding renal injury in mammals when administering an agent suspected to be associated with phospholipidosis.

### CROSS REFERENCE TO RELATED APPLICATION

This application claims the benefit of U.S. Provisional Application No. 60/384,728 filed May 31, 2002.

### FIELD OF THE INVENTION

This invention relates to methods for avoiding renal injury in mammals when administering an agent suspected to be associated with phospholipidosis.

### BACKGROUND OF THE INVENTION

Megalin, also known as gp330, is a 600 kD glycoprotein that is expressed in the renal proximal tubule epithelium, as well as in other tissues and cells, such as type II pneumocytes of the lung, epididymis, endometrium, epithelial cells of the inner ear, retinal epithelium, as well as on the embryonic yolk sac and neuroectoderm (see Christensen and Willnow (1999); and Zheng et al. (1994)). In the kidneys, megalin cfunctions as an endocytic receptor that is involved in the endocytic reabsorption of proteins within the proximal tubule prior to urine excretion. Reabsorbed proteins are subsequently degraded by means of lysosomes (see, for example, Maunsbach and Christensen (1992)).

Christensen and Willnow (1999) disclose that megalin mediates the reabsorption of three vitamin carrier proteins, vitamin D binding protein (DBP), retinol binding protein (RBP) and transcobalamin (TCII) and their associated vitamins, (OH) vitamin 25 D₃, vitamin A (retinol) and vitamin B₁₂, respectively.

Leheste et al. (1999) disclose that megalin knockout mice as well as patients with Fanconi syndrome having impaired proximal tubular function excrete increased amounts of protein and retinol in the urine.

International Application Publication No. WO 99/37757 discloses a method of detecting renal damage by measuring the level of cubilin in the urine of an individual suspected of having such damage.

A fundamental goal of pharmaceutical research and development is to determine and monitor, as early as possible, whether a proposed pharmaceutical agent presents a toxicity hazard, for example, to any of the vital organ systems of the body.

Phospholipidosis refers to the intracellular accumulation of undigested cell membrane material within lysosomes. Renal phospholipidosis can lead to severe kidney damage in animals and humans treated with certain agents, such as, for example, aminoglycoside antibiotics (see Molitoris (1997); and Sande and Mandell (1985)). Moestrup et al. (1993) disclose that polybasic drugs, including aminoglycosides, bind to megalin. Nagai et al. (2001) have suggested that megalin is involved in the renal cortical accumulation of aminoglycosides.

Girton et al (2002) report that the incidence of nephrotoxicity caused by aminoglycosides, has not changed substantially in the last two decades. It has remained at about 20% for short-term aminoglycoside (i.e., less than 14 days) and approaches 50% when longer-term treatment (i.e., greater 14 days) are required.

Conventional methods currently available for determining whether an agent causes phospholipidosis include detecting and quantifying phospholipid excretion in the urine. Such methods have the disadvantage of detecting the effects of phospholipidosis mostly after renal damage has already occurred. There exists a need for reliable methods of detecting phospholipidosis before renal damage occurs.

### SUMMARY OF THE INVENTION

One aspect of this invention provides methods for early detection of phospholipidosis comprising:
collecting a first urine sample from a mammal and quantifying the concentration of an endogenous megalin binding ligand therein;
administering an agent suspected to be associated with phospholipidosis to a mammal;
collecting a second urine sample from a mammal and quantifying the concentration of an endogenous megalin binding ligand therein; and
comparing the quantity of endogenous megalin binding ligand in said first urine sample and said second urine sample.

Another aspect of this invention provides methods for avoiding renal injury comprising:
administering a first quantity of an agent suspected to be associated with phospholipidosis to a mammal;
quantifying the concentration of an endogenous megalin binding ligand in the urine of said mammal;
administering a second quantity of said agent to said mammal, wherein said second quantity is less than a quantity that increases the level of said endogenous megalin binding ligand in the urine of said mammal.

An additional aspect of this invention is methods of avoiding renal injury comprising:
(a) collecting a first urine sample from a mammal and quantifying the concentration of an endogenous megalin binding ligand therein;
(b) administering to said mammal a first quantity of an agent suspected to be associated with phospholipidosis; and
(c) collecting a second urine sample from said mammal and quantifying the concentration of said endogenous megalin binding ligand therein; and
(d)(i) administering to said mammal a second quantity of said agent in an amount equal to or more of said first quantity, provided the concentration of said endogenous megalin binding ligand in said second urine sample is not higher than the concentration of said endogenous megalin binding ligand in said first urine sample, or;
(ii) administering to said mammal a second quantity of said agent in an amount that is less than said first quantity, provided the concentration of said endogenous megalin binding ligand in said second urine sample is higher than the concentration of said endogenous megalin binding ligand in said first urine sample.

Another aspect of this invention is methods of avoiding renal injury comprising:
collecting a first urine sample from a mammal and quantifying the concentration of an endogenous megalin binding ligand therein;
administering to said mammal an agent suspected to be associated with phospholipidosis; and
collecting a second urine sample from said mammal and quantifying the concentration of said megalin binding ligand therein; and
characterizing said mammal as having the attribute of being likely to develop phospholipidosis from the administration of said agent. Preferably said characterizing of said mammal comprises recording the identity of the mammal and said attribute in an information recording media. Preferrably said recording media is selected from magnetic media, optical media and paper media.

In a preferred embodiment of this invention said endogenous megalin binding ligand is selected from divalent calcium cations; an apolipoprotein; parathyroid hormone; insulin; β₂-microglobulin; epidermal growth factor; prolactin; lysozyme; cytochrome c; thyroglobulin; plasminogen; lactoferrin; receptor-associated protein; PAI-1; PAI-1-urokinase; PAI-1-tissue-type plasminogen activator; retinol; retinol binding protein; retinol-retinol binding protein complex; transcobalamin; 25-(OH) vitamin D₃; vitamin D₃ binding protein; 25-(OH) vitamin D₃-vitamin D₃ binding protein complex; vitamin B₁₂; transcobalamin; vitamin B₁₂-transcobalamin complex; albumin; and cubulin. In a more preferred embodiment, said endogenous megalin binding ligand is selected from retinol and albumin. In an even more preferred embodiment said endogenous megalin binding ligand is retinol.

In another preferred embodiment of the invention, said mammal is selected from rat, mouse, dog and a primate. In a more preferred embodiment, said mammal is a human.

In a further preferred embodiment,
said characterizing of said mammal comprises recording the identity of the mammal and said attribute in an information recording media.

“Megalin” refers to a protein that is expressed in the renal proximal tubule of a mammal, and whose cDNA encoding sequence has at least a 75% nucleotide identity with either the human megalin cDNA sequence having gene accession number U04441 disclosed in Korenberg, J. R. et al. (1994), gene accession number U33837 disclosed in Hjalm, G., et al. (1996)) or the rat megalin cDNA sequence having gene accession number L34049) disclosed in Saito et al. (1994.

“Megalin binding ligand” means: (1) a substance that binds with megalin, (2) a substance that is incorporated into a cell by endocytosis by a mechanism that is mediated by megalin or (3) a substance that itself binds to a substance described in (1) or (2) of this definition. The term “endogenous megalin binding ligand” means a megalin binding ligand that originates or is produced within a mammal.

“Hybridization” refers to the process by which a polynucleotide strand anneals with a complementary nucleotide sequence through base pairing under defined hybridization conditions. Hybridization is an indication that two nucleic acid sequences share a degree of compliment identity. Hybridization complexes form under permissive annealing conditions and, depending upon the degree of compliment identity, may remain hybridized after the “washing” step(s). The washing step(s) is particularly important in determining the stringency of the hybridization process, with more stringent conditions allowing less non-specific binding (i.e., less binding between pairs of nucleic acid strands that are not perfectly matched). Permissive conditions for annealing of nucleic acid sequences are routinely determined by one of ordinary skill in the art and may be consistent among hybridization experiments, whereas wash conditions may be varied among experiments to achieve the desired stringency, and therefore hybridization specificity.

“Moderately stringent conditions” means conditions for hybridization and washing under which nucleotide sequences encoding a peptide at least about 75% nucleotide identity to each other typically remain hybridized to each other. Such stringent conditions are known to those skilled in the art and can be found in Ausubel et al. (2001). For example, moderately stringent hybridization conditions refers to hybridization to filter or subtrate-bound polynucleotide in 0.5 M NaHPO₄, 7% SDS, 1 mM EDTA at 65° C., and washing in 0.2×SSC, 0.1% SDS at about 45° C.

“Nucleotide identity” means the sequence alignment of a nucleotide sequence calculated against another nucleotide sequence, e.g. the nucleotide sequence of human megalin. Specifically, the term refers to the percentage of residue matches between at least two nucleotide sequences aligned using a standardized algorithm. Such an algorithm may insert gaps in the sequences being compared in a standardized and reproducible manner in order to optimize alignment between the sequences, thereby achieving a more meaningful comparison. Percent identity between nucleotide sequences is preferably determined using the default parameters of the CLUSTAL W algorithm as incorporated into the version 5 of the MEGALIGN™ sequence alignment program. This program is part of the LASERGENE™ suite of molecular biological analysis programs (DNASTAR, Madison Wis.). CLUSTAL W is described in Thompson 1994).

“Nucleotide sequence” and “polynucleotide” refer to DNA or RNA, whether in single-stranded or double-stranded form. The term “complimentary nucleotide sequence” refers to a nucleotide sequence that anneals (binds) to a another nucleotide sequence according to the pairing of a guanidine nucleotide (G) with a cytidine nucleotide (C) and adenosine nucleotide (A) with thymidine nucleotide (T), except in RNA where a T is replaced with a uridine nucleotide (U) so that U binds with A.

The following abbreviations are used herein:

“ACS” means American Chemical Society; “° C.” means degrees centigrade; “cDNA” means complimentary DNA; “DBP” means vitamin D binding protein; “DMSO” means dimethyl sulfoxide; “DNA” means deoxyribonucleic acid; “EDTA” means ethylenediamine tetra-acetic acid; “ELISA” means enzyme-linked immunosorbent assay; “HPLC” means high pressure liquid chromatography; “kg” means kilogram; “mg” means milligram; “mL” means milliliter; “nm” means nanometer(s); “μm” means microgram; “nM” means nanomolar; “NMR” means nuclear magnetic resonance; “PCR” means polymerase chain reaction; “PAI-1” means plasminogen activator inhibitor 1; “RIA” means radioimmunoassay; “RNA” means ribonucleaic acid; “mRNA” means messenger RNA; “RBP” means retinol binding protein; “SDS” means sodium dodecyl sulfate; “SSC” means saline saline citrate; “TCII” means transcobalmin; “THF” means tetrahydrofuran; “v/v” means volume/volume.

### BRIEF DESCRIPTION OF THE DRAWINGS

FIG. 1 is an electron-micrograph showing phospholipid inclusions or myelin figures in rat proximal tubule following four days of treatment with 100 mg/kg of the aminoglycoside, gentamicin, according to the procedure in Example 1.

FIG. 2 is an electron-micrograph showing phospholipid inclusion as in FIG. 1, but with increased magnification.

FIG. 3 shows the results of Sudan black staining of the rat kidney tissue treated as in FIG. 1.

FIG. 4 illustrates normal rat kidney histology.

FIG. 5 shows early lesions formation in rat kidney tissue treated as in FIG. 1.

FIG. 6 shows extensive kidney tissue damage in rat kidney tissue following nine days of treatment with 100 mg/kg of the aminoglycoside, gentamicin, according to the procedure in Example 1.

### DETAILED DESCRIPTION OF THE INVENTION

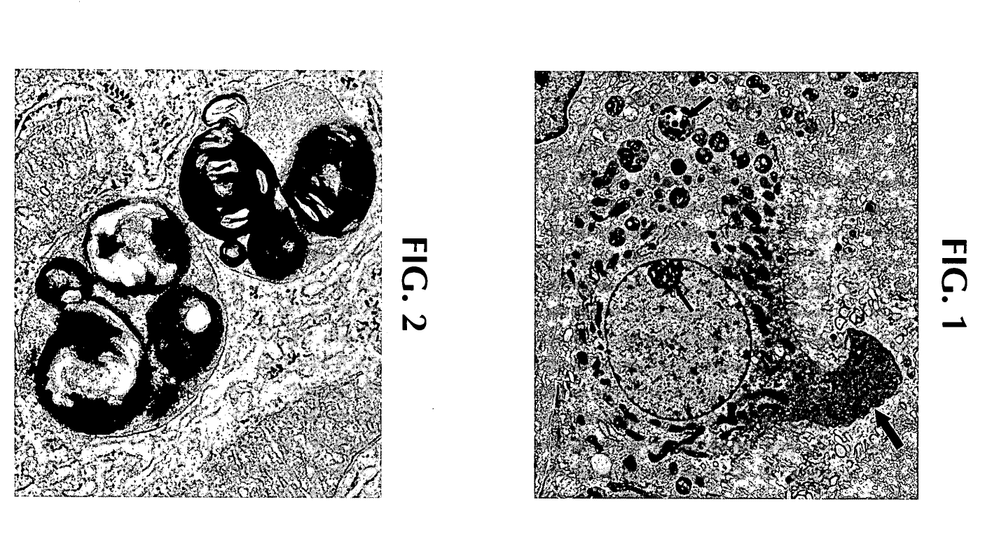

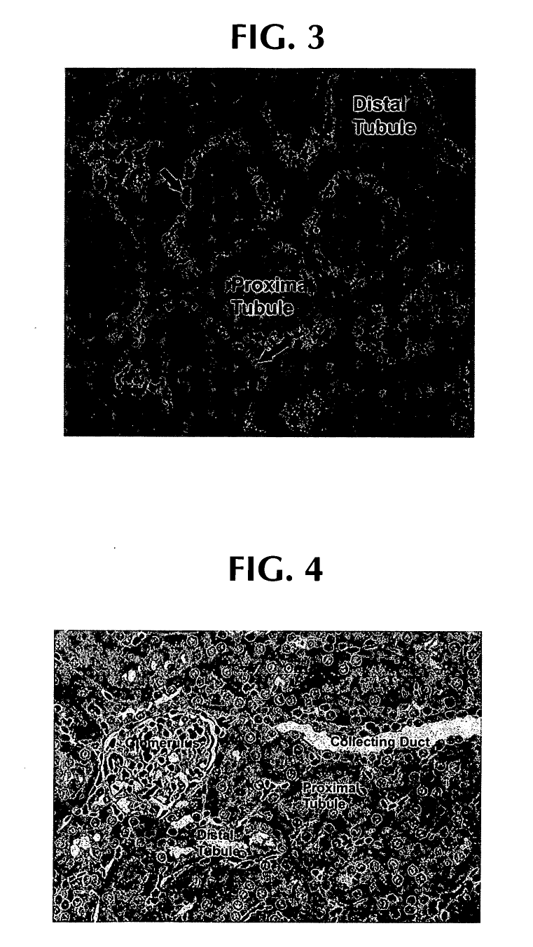

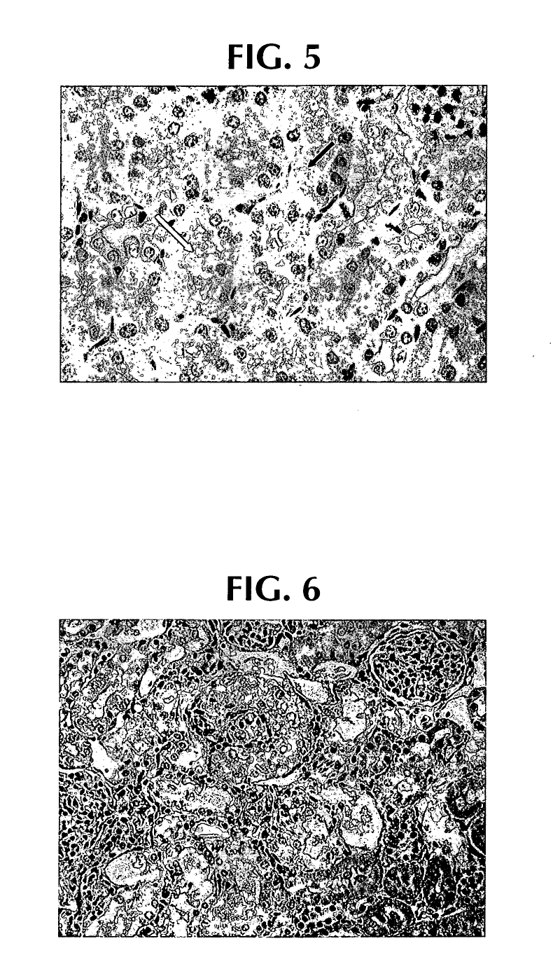

The physiological events that characterize the onset of phospholipidosis are well known to those with skill in the art (see, for example, Girton (2002)). Polybasic drugs such as the aminoglycoside, gentamicin, are incorporated in the cell through endocytosis by a mechanism that is mediated by megalin (Moestrup et al. (1995)). Following endocytosis, the aminoglycoside accumulates in lysosomes. The lysosomes eventually swell with excess lipid debris. When visualized using electron-microscopy, the accumulated lipid appear as phospholipid inclusions, or myelin figures, within the lysosomes (Kosek et al. (1974)) (see FIG. 1 and FIG. 2 below). The phospholipid inclusions may also be visualized by staining with Sudan black as described, for example, in Presnell and Schreibman (1997) (see FIG. 3 below). The damage to renal tissue from phospholipidosis may be visualized by histological examination using hematoxylin and eosin staining (H & E) of the renal tissue damaged through phospholipidosis. The damage that is evident includes tubular degeneration, vacuolation and eventually, in advanced stages of damage, necrosis of the renal proximal tubule (see FIG. 5 and FIG. 6 below).

This invention is based, in part, on the observation that, when a mammal is treated with a phospholipidosis-causing agent (e.g., gentamicin) there is an increase in the excretion of endogenous megalin binding ligands and that these ligands are detectable earlier in time than the time that renal injury is first seen to occur. The invention is particularly useful, for example, in the clinical setting as methods of early monitoring for the onset of phospholipidosis during the treatment of patients with potentially phospholipidosis-causing agents.

Such early monitoring methods may be performed according to the methods of the invention by determining the urinary excretion of an endogenous megalin binding ligand, such as, for example, retinol or albumin following administration of a potentially phospholipidosis-causing agent. If the level of endogenous megalin binding ligand increases following the administration, the dosage of the agent may be reduced in subsequent administrations or stopped altogether, thereby avoiding renal injury. In a preferred embodiment, the base level of the endogenous megalin binding is measured prior to the first administration of the potentially phospholipidosis-causing agent. The base level may then be compared to the level of the megalin binding ligand following administration of the agent in order to measure the increase, if any.

Megalin functions as an endocytic receptor, involved in the endocytic reabsorption of proteins within the proximal tubule of the kidney prior to urine excretion. While not wishing to be bound by any particular theory or mechanism, it is proposed that the mechanism by which phospholipidosis-causing agent result in increased excretion of endogenous megalin binding ligands involves two related modes of action. First, the phospholipidosis-causing agent binds to megalin, thereby displaces the endogenous megalin binding substrate. As a result, the endogenous substrate is not resorbed by endocytosis and is excreted in the urine. Second, it is proposed that the toxic agent impedes by an unknown mechanism the recycling of megalin from the endosomes back to the plasma membrane. As a result, less megalin is available for binding and endocytic reabsorption of the endogenous megalin binding ligands and this too contributes to increases ligand excretion.

As shown by Tables 1, 2 and 3 below, a significant increase in retinol and albumin excretion occurs after a single dose of gentamicin at 100 mg/kg. By comparison, as illustrated in Table 3 below, histopathologic effects do not begin to appear until day four. Similarly, as illustrated in Table 3, results from Sudan black staining show slight staining after a single dose of gentamicin (100 mg/kg). Significant Sudan black staining is not observed until day four.

TABLE 1
Urinary Retinol Concentrations In Rats.

| D s Gentamicin | Day 1 | Day 4 | Day 9 |
|---|---|---|---|
| 0 mg/kg | 72.15 nM | 73.99 nM | — |
| 100 mg/kg | 123.57 nM* | 131.08 nM* | 2457.13 nM* |

Concentration levels are calculated as the mean retinol concentration as measure for eight animals per dosage level. Dosing is performed according to the procedure in Example 1.
*Level of significance p < 0.05 (significance calculated using SigmaStat for Windows Version 2.03 Build 2.03.0 by SPSS Inc. (Chicago, IL)).

Concentration levels are calculated as the mean retinol concentration as measure for eight animals per dosage level. Dosing is performed according to the procedure in Example 1. *Level of significance p<0.05 (significance calculated using SigmaStat for Windows Version 2.03 Build 2.03.0 by SPSS Inc. (Chicago, Ill.)).

TABLE 2
Urinary Albumin Concentrations In Rats.

| Dose Gentamicin | Day 1 | Day 4 | Day 9 |
|---|---|---|---|
| 0 mg/kg | 81 μg/mL | 123 μg/mL | 95 μg/mL |
| 100 mg/kg | 163 μg/mL* | 354 μg/mL* | 1184 μg/mL* |

Concentration levels are calculated as the mean albumin concentration as measure for eight animals per dosage level.
*Level of significance p < 0.05 (significance calculated using SigmaStat for Windows Version 2.03 Build 2.03.0 by SPSS Inc. (Chicago, IL)).

Concentration levels are calculated as the mean albumin concentration as measure for eight animals per dosage level. *Level of significance p<0.05 (significance calculated using SigmaStat for Windows Version 2.03 Build 2.03.0 by SPSS Inc. (Chicago, Ill.)).

TABLE 3
Results of Histopathology of Rat Kidney.

| | | Day 1 | | Day 4 | | Day 9 | |
|---|---|---|---|---|---|---|---|
| D se Gentamicin mg/kg | | 0 | 100 | 0 | 100 | 0 | 100 |
| Total Number of Animals | | 8 | 8 | 8 | 8 | 8 | 8 |
| No significant findings | | 5 | 4 | 2 | | 5 | |
| Chronic Progressive | slight | 3 | 1 | 4 | 2 | 2 | |
| Nephropathy | | | | | | | |
| Hyaline droplets | slight | | 3 | 4 | 3 | 1 | 1 |
| | mild | | | | 1 | | 4 |
| Focal fibrosis | | | | 1 | | | |
| with tubular dilatation | | | | | | | |
| Tubular vacuolation | slight | | | | 3 | | 1 |
| | mild | | | | 3 | | 2 |
| | moderate | | | | | | 4 |
| Tubular | slight | | | | 6 | | |
| degeneration (1) | mild | | | | 2 | | |
| Tubular degeneration/ | slight | | | | | | |
| regeneration (2) | mild | | | | | | |
| | moderate | | | | | | |
| Tubular degeneration/ | marked | | | | | | 6 |
| necrosis/regeneration (3) | severe | | | | | | 2 |

(1) Brush border loss, occasionally vacuolated and/or necrotic cells.
(2) Brush border loss, occasionally vacuolated and/or necrotic cells; regenerating cells are present within affected tubules or as complete tubules.
(3) Brush border loss and flattening of the epithelium with numerous necrotic cells (present as part of tubule or within the lumen); frequent complete denudation of tubules with only the basement membrane remaining. Regeneration is a prominent feature, either within affected tubules or as separate tubules.

(1) Brush border loss, occasionally vacuolated and/or necrotic cells.
(2) Brush border loss, occasionally vacuolated and/or necrotic cells; regenerating cells are present within affected tubules or as complete tubules.

(3) Brush border loss and flattening of the epithelium with numerous necrotic cells (present as part of tubule or within the lumen); frequent complete denudation of tubules with only the basement membrane remaining. Regeneration is a prominent feature, either within affected tubules or as separate tubules.

TABLE 4
Results of Sudan Black Staining of Rat Kidney for Phospholipids

| | | |
|---|---|---|
| Day 1 | Day 4 | Day 9 |

| Dose Gentamicin mg/kg | 0 | 100 | 0 | 100 | 0 | 100 |
|---|---|---|---|---|---|---|
| Number of animals | 8 | 8 | 8 | 8 | 8 | 8 |
| N/A | 1 | | | | | |
| negative | 7 | 3 | 8 | | 8 | |
| slight | | 5 | | | | |
| slight-mild | | | | | | |
| mild | | | | 1 | | 1 |
| mild-moderate | | | | 2 | | 1 |
| moderate | | | | 2 | | 2 |
| moderate-marked | | | | 3 | | 3 |
| marked | | | | | | 1 |

Methods for identifying potential phospholipidosis-causing agents are known by those with skill in the art. For example, in vitro assays to identify potential phospholipidosis-causing agents have been described in Carrier et al. (1998), Carlier et al. (1983) and Carlier et al. (1984). Also, assays using cultured cells for predicting whether an agent may cause phospholipidosis have been described (see, for example, Oshima (1986) describing an assay using skin fibroblasts). Such assays may help to determine whether an agent has the chemical and/or physical properties to cause phospholipidosis once the agent is incorporated into lysosomes. In addition, methods have recently been described using metabonomics to identify potential phospholipidosis-causing agents (see, for example, Anthony et al. (2002)). Furthermore, standard histopathological methods may be used to examine tissues from animals treated with a test agent to determine if the agent causes renal phospholipidosis.

As those with skill in the art will appreciate based upon the present description, any mammal that expresses megalin in its kidneys may be used as a subject in carrying out the methods of this invention. Methods for identifying such a mammal will be apparent to those with skill in the art, and may include, for example, DNA-DNA or DNA-RNA hybridizations, polymerase chain reaction (PCR) amplification, and protein bioassay techniques which include membrane-, solution- and/or chip-based technologies for the detection and/or quantification of nucleotide or amino acid sequences. Such methods and techniques are described, for example, in Ausubel et al. (2001). Megalin can also be detected in mammal tissue by immunohistochemistry and immunocytochemistry methods as described in Kerjaschiki et al. (1984).

In a preferred method of identifying mammals expressing megalin in the kidneys, Northern blot analysis of total RNA isolated from kidney tissue of the mammal is performed using a hybridization probe, e.g., radiolabelled, single-stranded polynucleotide, having a sequence complementary to the human megalin cDNA sequence (gene accession number U04441, disclosed in Korenberg et al. (1994) and U33837, disclosed in Hjälm et al. (1996)) or the rat megalin cDNA sequence (gene accession number L34049, disclosed in Saito et al. (1994). Preferably, the polynucleotide probe is about 35 nucleotides in length and hybridization is performed under stringent conditions. In an alternative preferred embodiment, microarrays may be used to identify mammals that express megalin in their kidneys using methods known in the art (see, for example, Aigner et al. (2001); U.S. Pat. No. 5,965,352; Schena et al. (1995-A); DeRisi et al. (1996); Shalon et al. (1996); and Schena et al. (1995-B); U.S. Pat. No. 5,474,796; Schena, M., et al. (1996); WO 95/251116; WO 95/35505; Heller et al. (1997); and U.S. Pat. No. 5,605,662). The microarrays used in such a method would similarly contain one or more polynucleotide probes having a sequence that is complementary to the human or rat megalin cDNA sequences that are known.

In the utilization of such methods, excreted urine of a test subject is collected, and, optionally, concentrated. General methods of collecting, storing and handling urine specimens are known to those with skill in the art, as exemplified in Example 1. Any statistically significant increase in the urine level of megalin binding ligand over a control will be indicative of displacement of endogenous ligands by the test agent and the accumulation of the myelin figures in lysosomes. Preferably, such increase is at least about 25%, more preferably at least about 50% and even more preferably at least about 75% over the control or over the pretreatment value. Statistical significance is calculated using SigmaStat for Windows Version 2.03 Build 2.03.0 by SPSS Inc. (Chicago, Ill.).

As described above, for the purposes of this invention the term “endogenous megalin binding ligand” is meant to include an endogenous primary substance that binds to megalin, as well as a secondary endogenous substance that binds to the primary megalin binding ligand when the primary megalin binding substance is bound to megalin. Those with skill in the art will appreciate based upon the present description, that the particular endogenous megalin binding ligand detected and measured in the practice of this invention will depend upon a number of factors, including, for example, the ability of the ligand to be readily detectable if present in excreted urine. A variety of megalin binding ligands are known to exist, including, for example, those listed in Table 2 of Christensen and Willnow (1999). Preferred endogenous megalin binding ligands in the practice of this invention include retinol and albumin. Additional endogenous megalin binding ligands may be identified by one or more of the methods described in Christensen et al. (1992), Christensen and Willnow (1999), Cui et al. (1996), Gburek et al. (2002), Hilpert et al. (1999), Kanalas and Makker (1991), Kounnas et al. (1992), Kounnas et al. (1993), Kounnas et al. (1995), Moestrup et al. (1993), Moestrup et al. (1995), Moestrup et al. (1996), Moestrup et al. (1998), Nykjaer et al. (1999), Orlando et al. (1992), Orlando et al. (1998), Stefansson et al. (1995-A), Stefansson et al. (1995-B), Willnow et al. (1992), Willnow et al. (1996) and Zheng et al. (1998).

It will be appreciated by those with skill in the art based upon the present description, that the method of detection and quantification of endogenous megalin binding ligand in the practice of the invention may include any of a number of available analytical tools. For example, such methods may include the use of HPLC, NMR, or by using standard immunoassay methods known in the art. Such immunoassays include, but are not limited to, competitive and non-competitive assay systems using techniques such as RIAs, ELISAs, “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

It is believed that one skilled in the art can, based on the present description, including the examples, drawings, and attendant claims, utilize the present invention to its fullest extent. The following Examples are to be construed as merely illustrative of the practice of the invention and not limitative of the remainder of the disclosure in any manner whatsoever.

The disclosures of all patents, applications, publications and documents, including brochures and technical bulletins, cited herein, are hereby expressly incorporated by reference in their entirety.

### EXAMPLES

A. Dosing of Mammals, Collection and Storage of Urine

Male Sprague Dawley rats (105-125 grams each) were purchased from Charles River (Kingston, N.Y.). Gentamicin sulfate, 100 mg/mL, was purchased from Fermenta Animal Health Co. (Kansas City, Mo., catalog #568-2589, 2417). Rats were dosed intramuscular, using a 25 gauge tuberculin syringe. The animals were housed individually prior to urine collection. For the urine collection, rats were transferred to individual metabolism cages. At all times throughout the study, the animals had free access to food and water. Forty-eight rats were divided equally into two dosage groups: 0 mg/kg (control) and 100 mg/kg gentamicin. Rats were dosed once per day for the duration of 1, 4 or 9 days. Urine was collected after one dose, four doses and nine doses. Each collection period was for 24 hours. Urine was collected, chilled to approximately 4° C. and protected from light. Total urine volume was measured at the end of each collection period prior to retinol analysis. The urine was stored at −80° C. and in darkness to protect the components of the urine from oxidation and degration until analysis.

B. Measurement of Retinol in Urine Samples

1. Chemicals and Reagents used in HPLC.

The acetonitrile, methanol, isopropanol and tetrahydrofuran (THF) were purchased from J. T. Baker (Phillipsburg, N.J.). The acetonitrile and methanol were HPLC grade and the isopropanol and THF were ACS reagent grade. The water in the mobile phase came from a Nanopure® water filtration system (Barnstead, Dubuque, Ind.). Retinol and retinol acetate standards were purchased from ICN Biomedicals (Costa Mesa, Calif.). The retinol palmitate was purchased from Sigma Chemicals (St. Louis, Mo.).

2. HPLC Separation Conditions.

The HPLC instrumentation consisted of a Hewlett-Packard Model 1090 Series II system (Hewlett Packard Company, Palo Alto, Calif.) equipped with a tertiary gradient pump, diode array detector, and a cooled auto-sampler. Hewlett-Packard Chemstation™ software (Rev. 5.02) was used for data collection and analysis. Absorbance data for all retinols was collected at a wavelength of 340 nm. All separations were carried out on a 15 cm×2.0 mm, 5μ particle size, Prodigy ODS (2) HPLC column (Phenomenex, Torrance, Calif.). The column was equipped with an inlet filter, 0.5μ×1.5 mm (Phenomenex, Torrance, Calif.). The mobile phase preparation consisted of the following: Solvent A prepared by combining 250 mL of THF with 750 mL of acetonitrile. Solvent B consisted of 100% acetonitrile. Solvent C was prepared by combining 990 mL of water with 10 mL of methanol. Each mobile phase solvent was mixed and vacuum filtered prior to being placed on the HPLC system under continuous sparging with a light stream of helium. The gradient program consisted of: 90% Solvent B and 10% Solvent C for the first minute, over the next minute, ramp to 100% Solvent B, and hold at 100% B for one minute. Then, in 0.1 min, switch to 90% Solvent A, 10% Solvent B. Hold at 90% Solvent A/10% Solvent B for five minutes, then re-equilibrate for 3 minutes at the starting conditions. Total program time was 11 minutes and flow rate was 0.5 mL/minute Under the above mentioned conditions, retention times were 2.4 min. (retinol), 3.4 minutes (retinol acetate, internal standard), and 7.2 minutes (retinol palmitate).

3. Standard and Sample Preparation

While handling any standards and/or samples, care was taken to minimize exposure to natural or fluorescent lights. All work was conducted under yellow lighting, where possible. Retinol palmitate stock solutions were made up in DMSO. Stock solutions of retinol and retinol acetate were made in acetonitrile. Working solutions for all standards were prepared by serial dilution with acetonitrile. All standards and samples were stored in the dark, at −80° C.

4. Urine or Plasma Extraction

Rat urine (1.5 mL) was introduced into a 15 mL glass conical centrifuge tube. The internal standard of retinol acetate, was added to the sample (100 μl) at concentration of 100 μM. After vortexing vigorously for 10 seconds, 1.5 mL of a mixture of an 80%/20% (v/v) of methanol/isopropanol was added. Following 10 seconds of mixing, 4.5 mL of hexane was added. After mixing for one minute, the sample was centrifuged to separate into two layers. The hexane was removed, placed in a clean 15 mL glass centrifuge tube and evaporated in a water bath at 27° C. under a light stream of nitrogen. The hexane extraction was repeated two more times. Once all the combined hexane extracts were dried, the residue was reconstituted in 30 μl of acetonitrile, and mixed vigorously. The sample was then transferred to an auto-sampler vial, and remained in the dark auto-sampler at approximately 7° C., until 15 μl was injected onto the HPLC system.

Hence, the above Examples illustrate the collection, storage and handling of urine and the measurement of the endogenous megalin binding ligand, retinol, from said urine, in the practice of this invention. These Examples are to be construed as merely illustrative of the practice of the invention and not limitative of the remainder of the disclosure in any manner whatsoever.

### Literature

Aigner, et al. (2001) Arthritis and Rheumatism 44, 2777-89.
Anthony, M. L. et al. (2002) Tox Sciences 66 (1-S): 198.
Ausubel, F. M. et al. (2001) Current Protocols in Molecular Biology, John Wiley & Sons, New York, N.Y.
Carrier, D. et al. (1998) Biochemistry. 37, 7589-7597.
Carlier, M. B. et al. (1983) Agents & Chemotherapy 23, 440-449.
Carlier M. B. et al. (1984) Archives of Toxicology. Supplement. 7, 282-285.
Chistensen, I. L. and Willnow, T. E. (1999) J. Am. Soc. Nephrol. 10, 2224-2236.
Cui, S. et al. (1996) Am. J. Physiol. 271, F900-F907.
DeRisi, et al. (1996) Nature Genetics 14, 457-460.
Gburek, J. et al. (2002) J. Am. Soc. Nephrol. 13, 423-430.
Girton, R. A. et al. (2002) Am. J. Physiol. Renal Physiol. 282, F703-F709.
Hjalm, G., et al. (1996) Eur. J. Biochem. 239, 132-137.
Heller, R. A. et al. (1997) Proc. Natl. Acad. Sci. USA 94,2150-2155.
Hilpert et al. (1999) J. Biol. Chem. 274, 5620-5625.
Kanals, J. J. and Makker, S. P. (1991) J. Biol. Chem. 266, 10825-10829.
Kerjaschiki, D., et al. (1984) J. Cell Biol. 98, 178A.
Korenberg, J. R. et al. (1994) Genomics 22, 88-93.
Kosek, J. C. et al. (1974) Lab. Invest. 30, 48-57.
Kounnas, M. Z. et al. (1992) J. Biol. Chem. 267, 21162-21166.
Kounnas, M. Z. et al. (1993) J. Biol. Chem. 268, 14176-14181.
Kounnas, M. Z. et al (1995) J. Biol. Chem. 270, 13070-13075.
Leheste, J. et al. (1999) Am. J. Pathol. 155, 1361-1370.
Maunsbach, A. B. and Christensen, E. I. (1992) Handbook of Physiology: Renal Physiology, Windhager, editor, New York, Oxford University Press, 42-207.
Moestrup, S. K. et al. (1993) J. Biol. Chem. 268, 16564-16570.
Moestrup, S. K. et al. (1995) J. Clin. Invest. 96, 1404-1413.
Moestrup, S. K. et al. (1996) Proc. Natl. Acad. Sci. USA 93, 8612-8617.
Moestrup, S. K. et al. (1998) J. Clin. Invest. 102, 902-909.
Molitoris, B. A. (1997) Curr. Opin. Nephrol. Hypertens. 6, 384-388.
Nagai, J. et al. (2001) Am. J. Physiol. Renal Physiol. 281, F337-F344.
Nykjaer, A. et al. (1999) Cell 96, 507-515.
Orlando, R. A. et al. (1992) Proc. Natl. Acad. Sci. USA 89, 6698-6702.
Orlando, R. A. et al. (1998) J. Am. Soc. Nephrol. 9, 1759-1766.
Oshima, M. et al. (1986) Journal of Biochemistry 100, 1575-1582.
Presnell J. K. and Schreibman M. P. (1997) Humanson's Animal Tissue Techniques, 5th edition, The John Hopkins University Press, Maryland, 250-251.
Saito, A. et al. (1994) Proc Natl Acad Sci USA 91, 9725-9729.
Sande, M. A. and Mandell, G. L. (1985) The Pharmacological Basis of Therapeutics, Gilman et al., editors, New York, Macmillan, 1150-1169.
Schena et al. (1995-A) Science 270, 467-470.
Schena, et al. (1995-B) Proc. Natl. Acad. Sci. USA 93, 10539-11286.
Schena, M., et al. (1996) Proc. Natl. Acad. Sci. USA 93, 10614-10619.
Shalon, et al. (1996) Genome Res. 6, 639-645.
Stefansson, S. et al. (1995-A) J. Cell Sci. 108, 2361-2368.
Stefansson, S. et al. (1995-B) J. Biol. Chem. 270, 19417-19421.
Thompson, J. D. et al. (1994), Nucleic Acids Research 22, 4673-4680.
Wilnow, T. E. et al. (1992) J. Biol. Chem. 267, 26172-26180.
Wilnow, T. E. et al. (1996) Proc. Natl. Acad. Sci. USA 93, 8460-8464.
Zheng, G. et al. (1994) J. Histochem. Cytochem. 42, 531-542.
Zheng, G. et al. (1998) Endocrinology 139, 1462-1465.

1. A method for early detection of phospholipidosis comprising:
collecting a first urine sample from a mammal and quantifying the concentration of an endogenous megalin binding ligand therein;
administering an agent suspected to be associated with phospholipidosis to a mammal;
collecting a second urine sample from a mammal and quantifying the concentration of an endogenous megalin binding ligand therein; and
comparing the quantity of endogenous megalin binding ligand in said first urine sample and said second urine sample.

2. A method for avoiding renal injury comprising:
administering a first quantity of an agent suspected to be associated with phospholipidosis to a mammal;
quantifying the concentration of an endogenous megalin binding ligand in the urine of said mammal;
administering a second quantity of said agent to said mammal, wherein said second quantity is less than a quantity that increases the level of said endogenous megalin binding ligand in the urine of said mammal.

3. A method for avoiding renal injury comprising:
(a) collecting a first urine sample from a mammal and quantifying the concentration of an endogenous megalin binding ligand therein;
(b) administering to said mammal a first quantity of an agent suspected to be associated with phospholipidosis; and
(c) collecting a second urine sample from said mammal and quantifying the concentration of said endogenous megalin binding ligand therein; and
(d)(i) administering to said mammal a second quantity of said agent in an amount equal to or more of said first quantity, provided the concentration of said endogenous megalin binding ligand in said second urine sample is not higher than the concentration of said endogenous megalin binding ligand in said first urine sample, or;
(ii) administering to said mammal a second quantity of said agent in an amount that is less than said first quantity, provided the concentration of said endogenous megalin binding ligand in said second urine sample is higher than the concentration of said endogenous megalin binding ligand in said first urine sample.

4. A method for avoiding renal injury comprising:
collecting a first urine sample from a mammal and quantifying the concentration of an endogenous megalin binding ligand therein;
administering to said mammal an agent suspected to be associated with phospholipidosis; and
collecting a second urine sample from said mammal and quantifying the concentration of said megalin binding ligand therein; and
characterizing said mammal as having the attribute of being likely to develop phospholipidosis from the administration of said agent.

5. A method of claim 1, 2, 3 or 4 wherein said endogenous megalin binding ligand is selected from divalent calcium cations; an apolipoprotein; parathyroid hormone; insulin; β₂-microglobulin; epidermal growth factor; prolactin; lysozyme; cytochrome c; thyroglobulin; plasminogen; lactoferrin; receptor-associated protein; PAI-1; PAI-1-urokinase; PAI-1-tissue-type plasminogen activator; retinol; retinol binding protein; retinol-retinol binding protein complex; transcobalamin; 25-(OH) vitamin D₃; vitamin D₃ binding protein; 25-(OH) vitamin D₃-vitamin D₃ binding protein complex; vitamin B₁₂; transcobalamin; vitamin B₁₂-transcobalamin complex; albumin; and cubulin.

6. A method of claim 1, 2, 3, or 4 wherein said endogenous megalin binding ligand is selected from retinol and albumin.

7. A method of claim 1, 2, 3, or 4 wherein said mammal is selected from rat, mouse, dog and a primate.

8. A method of claim 1, 2, 3, or 4 wherein said mammal is human.

9. A method of claim 4 wherein said characterizing of said mammal comprises recording the identity of the mammal and said attribute in an information recording media.

10. A method of claim 9 wherein said recording media is selected from magnetic media, optical media and paper media.

11. A method of claim 3 wherein said endogenous megalin binding ligand is selected from retinol and albumin and said mammal is human.

12. A method of claim 11 wherein said endogenous megalin binding ligand is retinol.
